(19) United States
(12) Patent Application Publication
(10) Pub. No.: US 2002/0007186 A1
(43) Pub. Date: Jan. 17, 2002

(54) Amniotomy glove

(76) Inventor: Helio Zapata, Skokie, IL (US)

Correspondence Address: HELIO ZAPATA, 4142 W. CLEVELAND, SKOKIE, IL, 60076, US

(21) Appl. No.: 09/950,233

(22) Filed: Sep. 11, 2001

Related U.S. Application Data
(63) Continuation-in-part of application No. 09/349,798, filed on Jul. 9, 1999.

(51) Int. Cl. A61B 17/46

(52) U.S. Cl. 606/125

(57) ABSTRACT

An ordinary latex glove, or surgeon's glove, incorporating a hook in the structure thereof. The hook is mounted on a structural base, forming a hook component which is mounted on the inner surface of the middle finger stall of the glove, with the hook extending through the wall of the stall to the exterior.

This is a continuation-in-part of my previous application, Ser. No. 09/349,798, filed Jul. 9, 1999, for the Amniotomy Glove.

### FIELD OF THE INVENTION

The invention resides in the field of Medicine, specifically, in the branch of Obstetrics where practitioners perform amniotomy procedures in which the amniotic membrane is ruptured artificially. This procedure is done to make the process of labor more effective.

Heretofore there has not been any means or instrumentalities for performing this step in a satisfactory manner.

### BACKGROUND OF THE INVENTION

Amniotomy, the artificial rupturing of the fetal membranes, is a routine practice in laboring patients. Amniotomy is also performed, whenever possible, as an integral part of labor induction. At present, amniotomy is often done with a rigid, inflexible plastic stick 10½ inches long, with a small, plastic hook at its narrower end. If seen under a magnifying glass, this small hook looks like a small, inverted falcon's beak. This instrument is ineffective in many cases for accomplishing its intended purpose. In order to perform an amniotomy with such a device, the cervix has to be in an advanced stage of effacement and should also be sufficiently dilated. When the cervix, a hollow tube of fibrous tissue (whose axis is normally at an angle to that of the vagina), is neither effaced nor dilated (similar to the long neck of an inverted bottle) the above-mentioned device is ineffective. That device cannot have access to an uneffaced cervix because of the curvature needed to overcome the angle between the intersecting axes of the vagina and the cervix.

The inability to perform an amniotomy in these extremely common circumstances, when the head is already engaged, becomes a frequent obstacle. The only alternative the physician has is to wait for several hours for the cervix to open and efface before that device can be used to break the amnion. Often times, the desired dilatation and effacement never take place in spite of many hours of prostaglandings or labor contractions. The process of labor has a statistically higher chance of success with amniotomy than without it. Not infrequently, a cesarean section is done for a dead fetus because all conventional means to deliver it vaginally failed and, in most of those cases, an amniotomy could not be done because of an uneffaced cervix. Another problem with using the previous device mentioned is that it causes apprehension in many patients when they see the physician approaching them with this sizable stick.

All these problems associated with the use of the above-mentioned device for amniotomy are solved quickly and successfully with the use of the device of the present invention which is an ordinary examining glove that has a small plastic hook at the tip of its middle finger. Regardless of the cervical effacement, its orientation or consistency, if the physician can insert the gloved middle finger into the cervix and reach the amnion, the process is easily accomplished. The only limitations to the present device, then, would be a closed cervix or one that cannot be reached at all with the middle finger.

In any case, to break the amnion it is necessary to utilize a hook or point to engage the fetal membrane and rupture it. Currently, amniotomy procedures use that instrument which is separate from the physician's examining glove and has to be manipulated with the opposite hand to break the fetal membrane.

### SUMMARY OF THE INVENTION

The device embodying the present invention includes a regular physician's glove, and a rupturing point incorporated directly in the structure of the glove.

A principal feature of the present invention is to incorporate a single hook or point in the stall of the glove receiving the middle finger, so that it is manipulated solely by that finger.

Another main feature is the provision of a special physical construction of the hook, and its mounting in the glove, to simplify the overall construction, and facilitate its manipulation in the use thereof, and assure its proper manipulation.

Still another important feature is to provide a construction, in the device referred to, which is of extremely simple nature, and enables the user to easily perform an amniotomy at any time even when the cervix admits only one finger.

### BRIEF DESCRIPTIONS OF THE INDIVIDUAL FIGURES OF THE DRAWINGS

FIG. 1 is a perspective view of a physician's glove in which the puncturing point is incorporated.

FIG. 2 is a perspective view of the glove on the hand from a different angle from that of FIG. 1.

FIG. 3 is an enlarged face view of certain fingers of the glove, one of which has the puncturing hook incorporated therein.

FIG. 4 is a perspective view of a component incorporating the puncturing hook point, that is itself incorporated in the glove.

FIG. 5 is an enlarged sectional view taken at line 5-5 of FIG. 3.

FIG. 6 is a sectional view of the expectant mother's anatomy showing use of the amniotomy device.

### DETAILED DESCRIPTION OF THE DRAWINGS

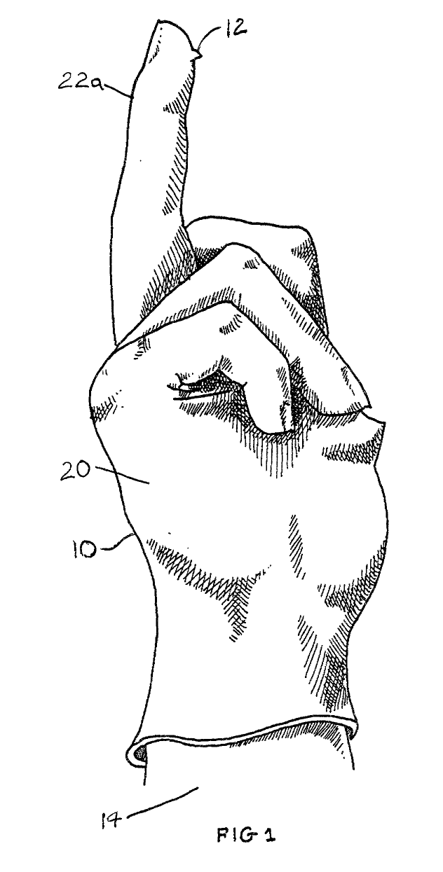

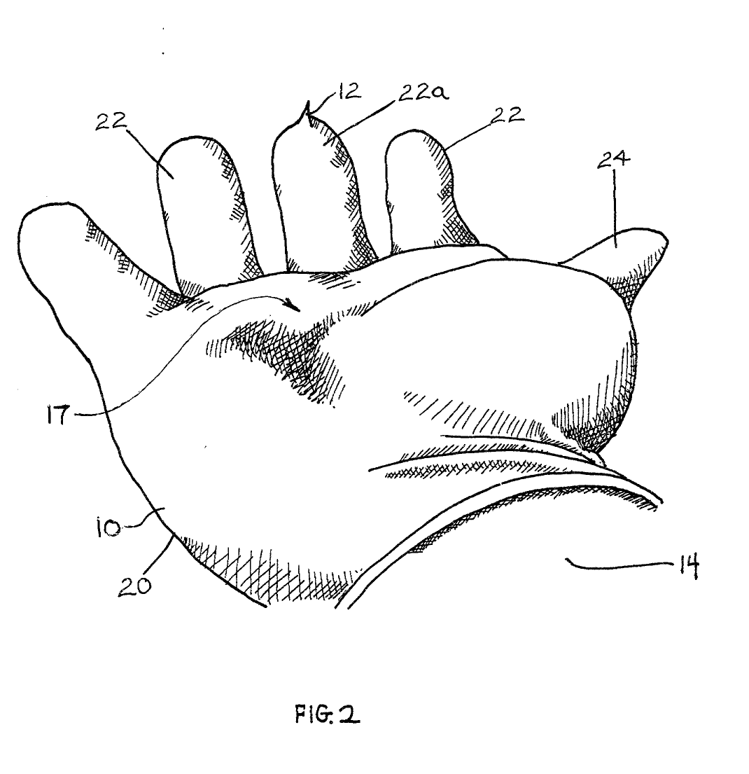

Attention is directed first to FIGS. 1 and 2 showing the amniotomy glove as a whole, and fitted on the hand of the user. The glove, as a whole is indicated at 10, and the puncturing hook is referred to here, indicated at 12, to be described in detail later.

The glove is shown fitted to the hand of the user, the hand itself being covered by the glove, and the wrist 14 being shown.

The glove 10 itself, is a standard and well-known physician's glove, made of latex. As it is well known, this latex material is of simple ply, elastic, yieldable, and stretchable. It is very flexible, and yields to the shaping of the hand but overall remains in place on the hand and does not shift on the hand to any great extent, this latter feature being of great significance in the manipulation of the hook or point.

The hand and the glove have a front side 17 (FIG. 2) or palm side, so referred to for convenience and in particular in describing the rupturing hook.

The glove has a main part 20, which fits over the main part of the hand, and finger stalls 22, and a thumb stall 24. The numeral 22a indicates the middle finger, this being of significance in connection with the location of the hook 12 and its functioning.

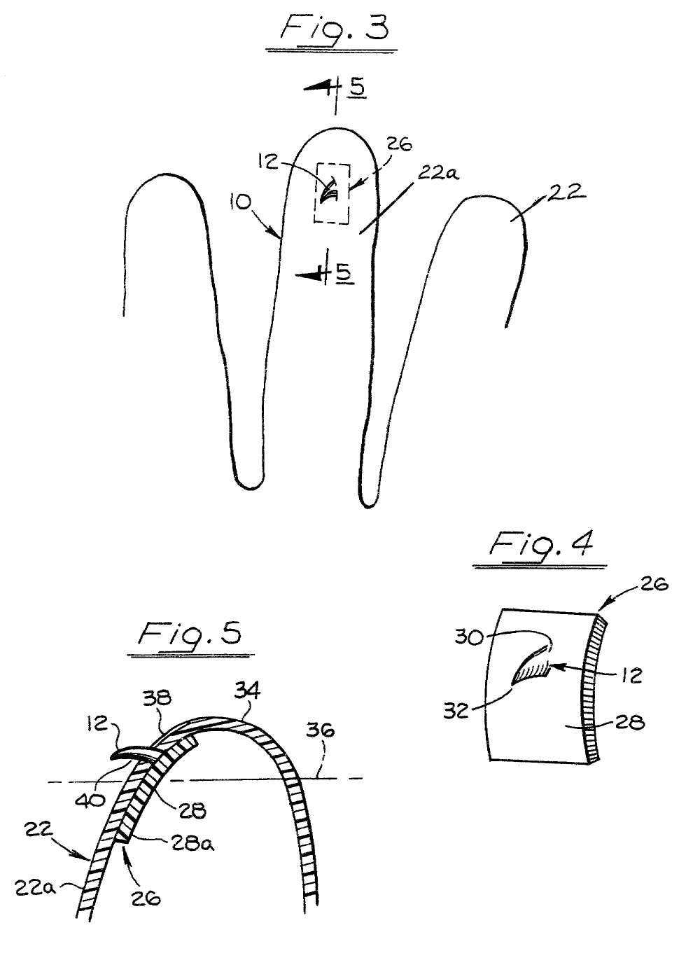

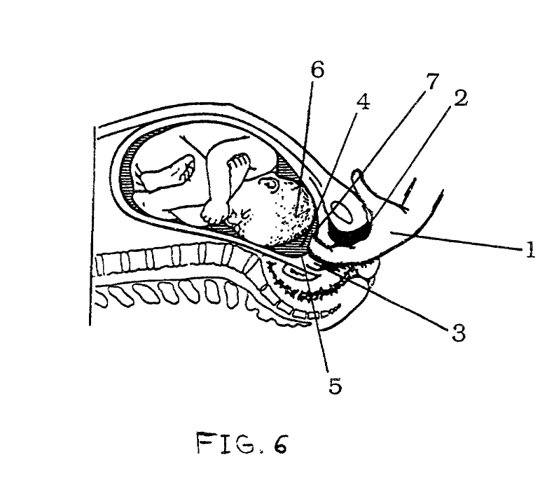

Reference is made to FIGS. 3-5 for a detailed description of the hook component which incorporates the hook. The hook component as a whole is indicated at 26 and includes a support base 28, of rigid material, a material that is less flexible than that of the glove. This support base is structurally in the form of a single ply sheet and is of a material such that it and the material of the glove are capable of being bonded securely together, as referred to again hereinbelow.

The hook is made of the same rigid, plastic material of the support base. The upper surface of the support base is bonded to the inner surface of the latex glove. Only the hook protrudes through the latex and remains outside the glove. The hook measures 2 mm high and 2 mm in its round base diameter.

The support base 28 is of physical size and proportions approximately as follows: length 15 mm, width 8 mm, and a thickness of 1 mm. The hook or point 12 is generally tapered, having a relatively broad base 30 and a relatively sharp point element 32.

This hook component is mounted inside the finger stall 22a as shown best in FIGS. 3 and 5. Each of the finger stalls is long, relative to its width; it is of generally tubular form and has a closed outer end 34 which approaches hemispherical form, having a base at line 36. This shape of the tip of the finger stall is of approximately the shape and the proportions described, although it need not be accurately so. Its shape is a result from being the closed end element of the tubular finger stall. The entire glove is impervious and without openings, except of course the entrance opening of the glove.

The point component 26 is located inside the finger stall 22a, receiving the middle finger (FIG. 5). This figure shows the structural support base 28 inside the finger stall, and applied to the inner surface of the stall, and secured thereto throughout the area of that base. As indicated above, it is secured in a thorough manner, being bonded to the surface of the finger stall throughout the area of the base.

The puncturing hook or element 12 is extended through the wall of the finger stall, as indicated at 38. It is so located that it is positioned outwardly, i.e. longitudinally to the finger, beyond the base line 36. In this position the upper or outer end of the base 28 is also beyond the base line 36, and its inner or lower end extends a greater distance below the base line 36.

Accordingly, the hook or point 12 is positioned at the outer end of the finger, and very close to the extreme outer end 34, for simplification of the maneuvering of the device.

It will be noted that the point 12 extends from the hand in the direction of the front or palm side of the hand, although located at the extreme outer end of the finger.

A sealing material is applied to the glove after positioning of the point component within the glove and securing the point to the glove. This sealing material is applied as shown at 40 in FIG. 5, it aids in solidifying the point so that it moves as a single element with that portion of the glove. In connection with this feature, the glove itself, as indicated above, does not shift relative to the middle finger stall but remains generally in a fixed location relative to the immediate area of the finger where the point is applied.

Accordingly, the point can be applied to the fetal membrane by maneuvering the user's middle finger.

The hook is very small compared with the hand, or any of the fingers or portions thereof. When the hand is about to be used, the middle finger, as well as the other fingers, are flexed, making a fist so that the hook is not visible to the patient. Accordingly, there is no fear developed in the patient, as would be in the case where an external article or device is used, and introduced in the vagina, which oftentimes produces fear in the patient. Because the hook is extremely small, it does not interfere with manipulating the finger, nor does it cause any harm to surrounding tissue.

The present invention allows amniotomy in the early stages of labor with an unfavorable cervix, thus increasing the chances of vaginal delivery at the outset of labor.

The middle finger can be easily inserted inside the cervix and the hook can be applied directly to the membrane. The user can touch the fetal membrane with the tip of the middle finger and then can move it back and forth in the most gentle manner. This maneuvering of the finger and the hook can be determined by the sense of touch. Since the tip of the hook is extremely close to the physician's hand and functions as a virtual extension of the user's middle finger, this sensing is very effective because the finger itself is extremely close to the membrane and thus the user can accurately determine the location and the positioning of the hook.

The hook's support base 28 is of substantial size relative to the hook, and thus provides the support strength referred to in the function of actually rupturing the membrane. As the finger is moved back and forth against the fetal membrane, the hook base 28 shifts with the glove, since the glove remains in a generally fixed location relative to the glove, and remains generally in a fixed position.

The feature of the hook, being effectively fixed on the finger, enables the user to introduce one finger, the middle finger, into the cervix and easily perform the procedure.

The hook is located closely adjacent to the outer end of the finger, as indicated above. The outer end of the finger is nearly of hemispheric shape and the hook is disposed outwardly from the base of that hemispheric shape. Thus, the hook is very near the tip end of the finger, but while it is so located, it also is curved inwardly (FIG. 1) in the direction of the palm or front side of the hand. Thus, since it is closely adjacent to the outer tip of the finger, it can be easily maneuvered into an active position, and since it is curved toward the palm side of the hand, the finger can be easily manipulated to apply the hook directly to the membrane and rupture it. This results in a complete maneuverability of the hook in substantially all directions, this being done by the immediate agency of the tip of the finger.

Another advantage is that in certain cases, it may be desired to perform the puncture at a more anterior position to prevent a sudden gush of amniotic fluid. This can be done very effectively and accurately because of the unique maneuverability of the middle finger inside the cervix and the potentiality of accurate sensing in a manner not possible in other methods or in the use of other devices or additional instrumentalities.

The axes of the cervix and the vagina are at a significant angle to each other (FIG. 4) and it is difficult if not impossible, in the case of an outside straight, rigid instrumentality, to move the hook back and forth against the membrane for performing the rupturing step when the cervix is not effaced. In the case of the present invention, that maneuver can be performed with great accuracy and ease, because the sharp angle between the cervix and vagina can be easily overcome by the natural curvature of the middle finger.

Because of the small size of the hook, and its direct application to the finger, the present invention may be utilized in early stages of labor, and even before labor begins, so long as the cervix admits one finger, an obvious advantage over such instrumentalities as an outside or exterior, straight, plastic hook.

I claim:

1. An amniotomy glove comprising,
a basic latex examining glove made up of a single ply of flexible and elastic material, for fitting on the hand of the user, and having a main part for fitting on the main portion of the hand, and stalls for fitting on the thumb and fingers,
the amniotomy glove including a hook component made up of a structural base support and a hook element,
the structural support base being positioned in the stall receiving the middle finger, and secured to the stall, and the hook element being secured to the structural support base and extended through the wall of the stall so that the hook is the only part that is outside the glove.

2. The amniotomy glove according to claim 1 wherein,
the material of the glove and the structural base are capable of being fused together to form an effectively integral piece.

3. The amniotomy glove according to claim 2 wherein,
the material of the basic glove and the hook element are capable of being fused together to form an effectively integral piece.

4. The amniotomy glove according to claim 3 wherein,
it is made up entirely of the basic glove, the structural base, and the hook element.

5. The amniotomy glove according to claim 1 wherein,
the basic glove has a palm side,
the structural base is in the form of a generally flat piece of rigid plastic material, secured to the concavity of the inner surface of the wall of said stall that is on the palm side of the basic glove, and closely adjacent to the outer end of the stall of the glove.

6. The amniotomy glove according to claim 1, wherein,
at least in the case of the middle finger, the outer end of the stall receiving the finger is of substantial hemispherical shape, and the hook is located at the convexity of said hemispherical shape.

7. An amniotomy glove according to claim 6 wherein,
said hook constitutes the only such hook, which is fixed to a structural base that is securely bonded to the latex material of the glove, combined with the flexibility of the middle finger of the user, enable the user to move the hook to a desired location and to engage the hook with the amniotic membrane and further manipulate it while in such engagement.

8. The amniotomy glove according to claim 7 wherein,
the user thereby moves the hook with a single finger, the middle finger.

9. The amniotomy glove according to claim 8 wherein,
the user is enabled to utilize the same single finger to enter the cervix and engage the amnion.

10. The Amniotomy Glove of claim 8 wherein, because of the structure recited, allows the user to perform an amniotomy, regardless of the effacement, consistency or position of the cervix, so long as it admits one finger.

11. The Amniotomy Glove according to claim 10 wherein, because of the structure recited, allows the user to easily overcome the sharp angle between the cervix and the vagina when the cervix is long and not sufficiently dilated.
